(19) United States
(12) Patent Application Publication
(10) Pub. No.: US 2024/0425853 A1
(43) Pub. Date: Dec. 26, 2024

(54) MODIFIED SMALL INTERFERING RNA MOLECULES WITH REDUCED OFF-TARGET EFFECTS

(71) Applicant: MICROBIO (SHANGHAI) CO. LTD., SHANGHAI (CN)

(72) Inventors: Yi-Chung Chang, SHANGHAI (CN); Chi-Fan Yang, SHANGHAI (CN); Hui-Yu Chen, SHANGHAI (CN); Chia-Chun Yang, SHANGHAI (CN)

(73) Assignee: MICROBIO (SHANGHAI) CO. LTD., SHANGHAI (CN)

(21) Appl. No.: 18/580,688

(22) PCT Filed: Jul. 21, 2022
(86) PCT No.: PCT/CN2022/107028

§ 371 Date: Jan. 19, 2024

(30) Foreign Application Priority Data
Jul. 22, 2021 (WO) PCT/CN2021/107862

(51) Int. Cl. C12N 15/113; A61P 35/00

(52) U.S. Cl. CPC C12N 15/113; A61P 35/00; C12N 2310/14; C12N 2310/315; C12N 2310/321; C12N 2310/322

(57) ABSTRACT

A modified small interfering RNA (siRNA) molecule comprising phosphorothioate (PS) intemucleotide linkages in the antisense strand for reducing off-target effects and methods and uses thereof. The siRNAs targeting Hypoxia Inducible Factor 1 Subunit Alpha (HIF1a) with high specificity and silencing efficiency.

### CROSS REFERENCE TO RELATED APPLICATIONS

This application claims the benefit of the filing date of International Patent Application No. PCT/CN2021/107862, filed on Jul. 22, 2021, the entire contents of which is incorporated by reference herein.

### BACKGROUND OF THE INVENTION

RNA interference (RNAi) is a process of sequence-specific post-transcriptional gene silencing that is mediated by small interfering RNAs (siRNAs). Considerable attention is given to the ability to influence the RNAi to specifically silence the expression of the target genes in order to achieve desired therapeutic effects.

The challenges facing siRNA therapeutics are significant. This is because the inherent properties of siRNAs, such as being polyanionic, vulnerability to nuclease cleavage make clinical application difficult due to poor cellular uptake and rapid clearance. In addition, the off-target effects that arise due to deleterious protein binding or mis-targeting of mRNA can further limit the siRNA therapy.

Accordingly, there is a growing need to develop potent siRNAs with strongly reduced off-target effects.

### SUMMARY OF THE INVENTION

The present disclosure is based, at least in part, on development of modified small interfering RNA (siRNA) molecules that show reduced off-target effect. Accordingly, provided herein are modified siRNAs having reduced off-target effects and uses thereof for silencing a target gene, e.g., those associated with a disease or disorder.

In some aspects, the present disclosure provides a modified small interfering RNA (siRNA) molecule, comprising a sense strand and an antisense strand. The antisense strand comprises phosphorothioate (PS) internucleotide linkages between nucleotides at positions 5 and 6 and/or between nucleotides at positions 6 and 7. The modified siRNA has reduced off-target effect as compared with the siRNA counterpart that has no PS internucleotide linkages between nucleotides at positions 5 and 6 and between nucleotides at positions 6 and 7. In some embodiments, the modified siRNA molecule may be associated with a targeting moiety.

In some embodiments, the antisense strand of the modified siRNA molecule may further comprise PS internucleotide linkages between nucleotides at positions 1 and 2 and/or between nucleotides at positions 2 and 3. Alternatively or in addition, the antisense strand of the modified siRNA molecule further comprises PS internucleotide linkages between the first and second nucleotides at the 3′ end and/or between the second and third nucleotides at the 3′ end.

In some embodiments, the antisense strand of the modified siRNA molecule is of 19-25 nucleotides in length. For example, the antisense strand of the modified siRNA molecule is of 21 nucleotides in length. In that case, the antisense strand of the modified siRNA molecule may further comprise PS internucleotide linkages between nucleotides at positions 19 and 20 and/or between nucleotides at positions 20 and 21.

In some embodiments, the modified siRNA molecule silences expression of a pathogenic gene, which optionally is a bacterial gene, a viral gene or a fungal gene. In other embodiments, the modified siRNA molecule silences expression of a disease gene. Exemplary disease genes include, but are not limited to, those involved in cancer, fibrosis, a metabolic disease, a cardiovascular disease, an immune disease, or an inheritance disorder.

In some examples, the disease gene may be involved in cancer. Specific examples include HIF1A, HIF2, IGF1R, VEGF, EREG, KRAS, ALK, BRAF, NRAS, STAT3, CDH2, KIFL1, PIK3CA, Src, RAS, RAF, and TP53. In some examples, the disease gene may be involved in fibrosis. Specific examples include HIF1A, HIF1B, HIF2, TGF-β1, and CTGF. In some examples, the disease gene may be involved in a metabolic disease or a cardiovascular disease. Specific examples include AGT, ApoC-III, and apoB. In some examples, the disease gene may be involved in an immune disease. Specific examples include GATA-3, CCR3, TGF-α1, IL-6, TNF-α, IFN-β, IL-1β, CCL2, and CCL10. In other examples, the disease gene may be involved in an inheritance disorder. Specific examples include apoB and PCSK9.

In other aspects, the present disclosure features a pharmaceutical composition comprising any of the modified siRNA molecules disclosed herein and a pharmaceutically acceptable carrier.

Further, provided herein is a method for silencing a target gene, comprising contacting the modified siRNA molecule or the pharmaceutical composition comprising the modified siRNA molecule and a pharmaceutically acceptable carrier with cells expressing the target gene. In some embodiments, the contacting step can be performed by administering the modified siRNA molecule or the pharmaceutical composition to a subject in need thereof.

In another aspect, provided herein is an interfering RNA that targets human hypoxia inducible factor 1 subunit alpha (HIF1α) (anti-HIF1a interfering RNA). The interfering RNA comprises a nucleotide sequence complementary to a target site in a HIF1α mRNA. The target site may comprise a nucleotide sequence of:

(a)
(SEQ ID NO: 1)
AGGCCACAUUCACGUAUAU;
(b)
(SEQ ID NO: 2)
UGAGGAAGUACCAUUAUAU;
(c)
(SEQ ID NO: 3)
CCGGUUGAAUCUUCAGAUA;
(d)
(SEQ ID NO: 4)
GCGCAAGUCCUCAAAGCAC;
(e)
(SEQ ID NO: 5)
AGGCCACAUUCACGUAUA;
or
(f)
(SEQ ID NO: 6)
UGAGGAAGUACCAUUAUA

In some embodiments, the target site in the HIF1α mRNA comprises the nucleotide sequence of AGGCCACAUUCACGUAUA (SEQ ID NO: 5). In other embodiments, the target site in the HIF1α mRNA comprises the nucleotide sequence of UGAGGAAGUACCAUUAUA (SEQ ID NO: 6).

In some embodiments, the anti-HIF1a interfering RNA is a siRNA comprising a sense strand and an antisense strand. In some instances, the antisense strand may be of 19-25 nucleotides in length. In examples, the sense strand and the antisense strand comprises the following nucleotide sequences, respectively: 5′-AGGCCACAUUCACGUAUAA-3′ (SEQ ID NO: 7) and 5′-UUAUACGUGAAUGUGGCCUGU-3′ (SEQ ID NO: 8). In other examples, the sense strand and the antisense strand comprises the following nucleotide sequences, respectively: 5′-UGAGGAAGUACCAUUAUAA-3′ (SEQ ID NO: (9) and 5′-UUAUAAUGGUACUUCCUC AAU-3′ (SEQ ID NO: 10).

In some embodiments, the antisense strand of an anti-HIF1a siRNA may comprise phosphorothioate (PS) internucleotide linkages between nucleotides at Positions 5 and 6 and/or between nucleotides at Positions 6 and 7. Such a modified siRNA has reduced off-target effect as compared with the siRNA counterpart that has no PS internucleotide linkages between nucleotides at Positions 5 and 6 and between nucleotides at Positions 6 and 7. In some instances, the antisense strand further comprises PS internucleotide linkages between nucleotides at Positions 1 and 2 and/or between nucleotides at Positions 2 and 3. Alternatively or in addition, the antisense strand further comprises PS internucleotide linkages between the first and second nucleotides at the 3′ end and/or between the second and third nucleotides at the 3′ end.

Any of the anti-HIF1a interfering RNAs disclosed herein may further comprises one or more modified nucleotides. For example, the anti-HIF1a interfering RNAs may comprise one or more modified nucleotides comprising 2′-fluoro, 2′-O-methyl, or a combination thereof.

In addition, the present disclosure features a pharmaceutical composition, comprising any of the anti-HIF1a interfering RNAs as disclosed herein and a pharmaceutically acceptable carrier.

In yet another aspect, the present disclosure features a method for suppressing expression of human HIF1α, the method comprising contacting an effective amount of any of the anti-HIF1a interfering RNAs disclosed herein with a cell that expresses human HIF1α. In some embodiments, the method comprises administering the effective amount of the interfering RNA or a pharmaceutical composition comprising such to a subject. In some examples, the subject is a human patient having or suspected of having a disease associated with HIF1α. Exemplary diseases associated with HIF1α include a cancer (e.g., a solid tumor), a heart disease (e.g., ischemic heart disease, or congestive heart failure), a lung disease (e.g., pulmonary hypertension, pulmonary fibrosis, or chronic obstructive pulmonary disease), a liver disease (e.g., acute liver failure, liver fibrosis, or liver cirrhosis), a kidney disease (e.g., acute kidney injury or chronic kidney disease), obesity, or diabetes.

Also within the scope of the present disclosure are pharmaceutical compositions comprising any of the modified siRNAs or the anti-HIF1a interfering RNAs for treating a target disease as disclosed herein, as well as uses of the modified siRNAs or the anti-HIF1a interfering RNAs for manufacturing a medicament for use in treating the target disease.

The details of one or more embodiments of the disclosure are set forth in the description below. Other features or advantages of the present disclosure will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a graph illustrating the off-target events caused by HIF1A siRNAs as assessed by genome-wide RNA sequencing. The tested siRNAs have PS internucleotide linkages at various positions as indicated by an asterisk (′*′). Numbers at the left side refer to down events; numbers at the right side refer to up events.

FIG. 2 is a graph illustrating the knockdown efficiency of HIF1A siRNAs having phosphorothioate (PS) internucleotide linkages at various positions as indicated.

FIGS. 3A and 3B include graphs showing in vivo effects of exemplary anti-HIF1A siRNA. FIG. 3A: Knockdown of HIF1A expression in human HepG2 xenograft mice. FIG. 3B: Inhibition of tumor growth in xenograft mice.

### DETAILED DESCRIPTION OF THE INVENTION

RNA interference or “RNAi” is a process in which double-stranded RNAs (dsRNA) block gene expression when it is introduced into host cells. (Fire et al. (1998) Nature 391, 806-811). One of the obstacles to RNAi therapy is the off-target effects (Seok et al (2018), Cell Mol. Life Sci. 75, 797-814). Short interfering RNA molecules (siRNA) are commonly used in RNAi to inhibit expression of a target gene.

siRNAs are double-stranded RNAs, an anti-sense strand and a sense strand, which contain complementary sequences and form the double-stranded structure. At least part of the anti-sense strand is complementary to a region within a target mRNA for blocking expression of the mRNA via RNAi. Each strand of a siRNA molecule may have 19-23 nucleotides. In some instances, each strand may have phosphorylated 5′ ends and hydroxylated 3′ ends. In some instances, the anti-sense strand may have a couple overhanging nucleotides (e.g., 1 or 2). When the siRNA is transfected into a cell, it is incorporated into the RNA-induced silencing complex (RISC), which includes the core protein Argonaute (AGO). Subsequently, the siRNA is unwound into single-stranded RNAs. Following which, the antisense strand remains associated with AGO to form an active RISC, whereas the sense strand is degraded. The antisense strand forms base-pairings with a target transcript (mRNA), and AGO cleaves the target to silence its function (gene expression).

Off-target effect is one potential problem associated with siRNA therapeutics. Therefore, developing siRNAs with reduced off-target effects is highly desirable towards a highly potent and safe RNAi therapy.

The present disclosure is based, at least in part, on the development of modified siRNA molecules, in which the common phosphodiester backbone linkage at certain nucleotide positions within the antisense strand is replaced with the phosphorothioate (PS) linkage (also referred as ‘PS bond’). In this substitution, a non-bridging phosphate oxygen atom is substituted with a sulfur atom to create the PS linkage between the nucleotides. Specifically, the PS modification is introduced in the seed region of the antisense strand of the siRNA molecule. For example, the PS modification can be introduced between nucleotides at one or more of Positions 5 to 8 in the antisense strand of the siRNA molecule. The modified siRNAs disclosed herein would have substantially reduced off-target effects and would also be expected to be more resistant to nucleases as compared with a counterpart nucleic acid (having the same nucleotide sequence) with no PS bonds at the defined positions.

Unless otherwise explicitly stated, the position of a nucleotide in a nucleic acid chain as disclosed herein refers to the position from the 5′ end of that nucleic acid chain, i.e., with the 5′ end nucleotide as Position 1.

I. Modified siRNA Molecules with Reduced Off-Target Effects

In some aspects, this present disclosure relates to modified small interfering nucleic acid molecules (siRNA) having reduced off-target effects relative to the same siRNA molecules that do not have the corresponding modifications.

(A) siRNA Molecules:

The disclosure relates to modified siRNA molecules, which are double-stranded RNAs capable of inducing gene silencing via the RNAi pathway against the target gene transcript and also having reduced off-target effects (against non-target gene transcripts).

The modified siRNA molecule comprises a sense strand and an antisense strand. The antisense strand comprises one or more phosphorothioate (PS) internucleotide linkages (also referred as ‘PS group’ or ‘PS bond’) in the seed region (Positions 5-8). The modified siRNA molecule has reduced off-target effect as compared to a siRNA counterpart that has no PS linkage at the respective nucleotide positions, e.g., by at least 30%, at least 40%, at least 50% or higher. Reduction of off-target effects can be determined via routine practice or by methods disclosed herein.

In some embodiments, the antisense strand in a modified siRNA disclosed herein may comprise a PS internucleotide linkage between nucleotides at Positions 5 and 6. Alternatively or in addition, the antisense strand in the modified siRNA may comprise a PS internucleotide linkage between nucleotides at Positions 6 and 7. Alternatively or in addition, the antisense strand in the modified siRNA may comprise a PS internucleotide linkage between nucleotides at Positions 7 and 8. In some examples, the antisense strand in the modified siRNA may comprise PS internucleotide linkages between nucleotides at Positions 5 and 6 and between nucleotides at Positions 6 and 7. In some examples, the antisense strand in the modified siRNA may comprise PS internucleotide linkages between nucleotides at Positions 5 and 6 and between nucleotides at Positions 7 and 8. In some examples, the antisense strand in the modified siRNA may comprise PS internucleotide linkages between nucleotides at Positions 6 and 7 and between nucleotides at Positions 7 and 8.

In some embodiments, the antisense strand in a modified siRNA disclosed herein may further comprise one or more PS internucleotide linkages at the 5′ end region, for example, between nucleotides at Positions 1 and 2, and/or between Positions 2 and 3. Alternatively or in addition, the antisense strand in a modified siRNA disclosed herein may further comprise one or more PS internucleotide linkages at the 3; end region, for example, between the first and second nucleotides at the 3′ end and/or between the second and third nucleotides at the 3′ end. For example, when an antisense strand contains 21 nucleotides, the PS internucleotide linkages at the 3; end region may be between the nucleotides at Positions 19 and 20 and/or between the nucleotides at Positions 20 and 21.

In some examples, the antisense strand in a modified siRNA as disclosed herein may contain PS internucleotide linkages within the seed region, e.g., between nucleotides at positions 5 and 6 and between nucleotides at positions 6 and 7, and at the 5′ end (e.g., 1 or 2) and/or the 3′ end (e.g., 1 or 2). In one specific example, the antisense strand contains (a) PS internucleotide linkages between nucleotides at Positions 5 and 6 and between nucleotides at Positions 6 and 7; (b), two PS internucleotide bonds at the 5′ end; and (c) two PS internucleotide bonds at the 3′ end.

In some examples, the antisense strand in a modified siRNA as disclosed herein may contain PS internucleotide linkages within the seed region, e.g., between nucleotides at positions 5 and 6, between nucleotides at positions 6 and 7, and between nucleotides at positions 7 and 8, and at the 5′ end (e.g., 1 or 2) and/or the 3′ end (e.g., 1 or 2). In one specific example, the antisense strand contains (a) PS internucleotide linkages between nucleotides at Positions 5 and 6 between nucleotides at Positions 6 and 7, and between nucleotides at Positions 7 and 8; (b), two PS internucleotide bonds at the 5′ end; and (c) two PS internucleotide bonds at the 3′ end.

In any of the modified siRNA molecules disclosed herein, the antisense strand may contain 15-30 nucleotides in length, e.g., 18-25 or 19-23 nts in length. In one example, the antisense strand includes 21 nts in length. In another example, the antisense strand includes 23 nts in length. The sense strand or a portion thereof is complementary (completely or partially) to the antisense strand or a portion thereof. In some instances, the sense strand has the same length as the antisense strand. In other instances, the sense strand is shorter than the antisense strand (e.g., by 1-5 nt such as by 1 nt, 2 nt, 3 nt, 4 nt, or 5 nt). In that case, the antisense strand may have overhang (e.g., 1-5 nts) at the 5′ end and/or at the 3′ end.

In one specific example, the antisense strand in a modified siRNA is 21 nts in length and the sense strand in the modified siRNA is 16 nts in length. The 5-nt overhang in the antisense strand can be located at its 3′ end. In another specific example, the antisense strand in a modified siRNA is 21 nts in length and the sense strand in the modified siRNA is 19 nts in length. The 2-nt overhang in the antisense strand can be located at its 3′ end.

Table 1 list exemplary siRNAs having exemplary PS internucleotide linkages in the seed region, and at the 5′ end and/or 3′ end. siRNAs having PS internucleotide linkages at the positions shown in the exemplary siRNAs listed in Table 1 are within the scope of the present disclosure.

### (B) Other Modifications

In addition to the PS internucleotide linkage modifications in the antisense strand of the modified siRNAs as disclosed herein, the antisense strand, the sense strand, or both of the modified siRNAs can further comprise other modifications such as sugar modifications, nucleobase modifications, backbone modifications, or a combination thereof. Such modifications may confer one or more desirable properties, for example, enhanced cellular uptake, improved affinity to the target nucleic acid, increased in vivo stability, enhance in vivo stability (e.g., resistant to nuclease degradation), and/or reduce immunogenicity.

In one example, the modified siRNAs disclosed herein (e.g., in the sense strand and/or antisense strand) may have a modified backbone at positions different from the PS internucleotide bonds, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In some examples, the modified siRNAs disclosed herein do not include any backbone modifications, except for the PS internucleotide bonds disclosed herein.

In another example, the modified siRNAs disclosed herein (e.g., in the sense strand and/or antisense strand) include one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C1 to C10 alkyl or C2 to C10 alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

Alternatively or in addition, the modified siRNAs disclosed herein (e.g., in the sense strand and/or antisense strand) include one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993. Certain of these nucleobases are particularly useful for increasing the binding affinity of the interfering RNA molecules to their targeting sites. These include 5-substituted pyrimidines, 6-azapyrimidines and N-2, N-6 and O-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Alternatively or in addition, the modified siRNAs disclosed herein (e.g., in the sense strand and/or antisense strand) may comprise one or more locked nucleic acids (LNAs). An LNA, often referred to as inaccessible RNA, is a modified RNA nucleotide, in which the ribose moiety is modified with an extra bridge connecting the 2′ oxygen and 4′ carbon. This bridge “locks” the ribose in the 3′-endo (North) conformation, which is often found in the A-form duplexes. LNA nucleotides can be used in any of the modified siRNAs disclosed herein. In some examples, up to 50% (e.g., 40%, 30%, 20%, or 10%) of the nucleotides in an interfering RNA are LNAs.

In some aspects, any of the modified siRNA molecules described herein may be conjugated to a ligand (targeting moiety) or encapsulated into vesicles that can facilitate the delivery of the modified siRNA to desired cells/tissues and/or facilitate cellular uptake. Suitable ligands include, but are not limited to, carbohydrate, peptide, antibody, polymer, small molecule, cholesterol and aptamer. For example, one or more GalNAc moieties (e.g., a tri-GalNAc moiety) may be used as the targeting moiety for delivering the modified siRNAs to liver cells.

### (C) Target Genes

The modified siRNA as disclosed herein is for use to suppress expression of a target gene, the transcript of which (mRNA) contains a region that is complementary to the antisense strand in the modified siRNA. Accordingly, the sequence of the antisense and sense strands can be designed based on the mRNA sequence of the target gene. In some instances, the antisense strand may be completely complementary to a target region within the mRNA of the target gene. In other instances, the antisense strand may be partially complementary to a target region within the mRNA of the target gene (e.g., contain one or more mismatches or gaps) as long as the level of complementarity is sufficient for base-pairing with the target region, which is within the knowledge of a skilled person in the art.

In some embodiments, the target gene of the modified siRNA disclosed herein is a pathogenic gene. For example, the target gene may be a gene of a pathogen, e.g., a virus, a bacterium, or a fungus. In other examples, the target gene is involved in a disease or disorder, for example, cancer, an immune disorder (e.g., an autoimmune disease), metabolic disorders or diseases, cardiovascular disorders or diseases and other inherited disorders or diseases.

In some examples, the modified siRNA silences expression of a target gene involved in cancer. Exemplary cancer-associated target genes include, but are not limited to, HIF1A, HIF2, IGF1R, VEGF, EREG, KRAS, ALK, BRAF, NRAS, STAT3, CDH2, KIFL1, PIK3CA, Src, RAS, RAF, and TP53.

In some examples, the modified siRNA silences expression of a target geen involved in fibrosis. Exemplary fibrosis-associated target genes include, but are not limited to, HIF1A, HIF1B, HIF2, TGF-β1, and CTGF.

In some examples, the modified siRNA silences expression of a target gene involved in a metabolic disease. Exemplary metabolic-associated target genes include, but are not limited to, AGT, ApoC-III, and apoB.

In some examples, the modified siRNA silences expression of a target gene involved in an immune disease (e.g., an autoimmune disease). Exemplary immune disease-associated target genes include, but are not limited to, GATA-3, CCR3, TGF-β1, IL-6, TNF-α, IFN-γ, IL-1β, CCL2, and CCL10.

### II. Interfering RNAs Targeting Human Hypoxia Inducible Factor 1 Subunit Alpha (HIF1α)

Hypoxia inducible factor-1A (HIF-1A) is the major transcription factor that has a prominent role in regulating cellular responses to hypoxia. Lyer et al., Genes Dev 1998, 12, 149-162. Under normoxic conditions, HIF-1a subunit is continuously synthesized and degraded by the ubiquitin-proteasome system. Under hypoxia conditions, HIF-1A is overly expressed in various cancers and regulates various genes involved in tumor growth, angiogenesis, chemotherapy tolerance, invasion and metastasis. Favaro et alGenome Med. 2011, 3:1-12; and Gonzalez et al., Nat. Rev. Endocrinol. 2018, 15, 21-32. HIF-1A was upregulated in hepatocellular carcinoma, and associated with hepatic capsular invasiveness and portal vein metastasis. Feng et al., Cell Mol. Biol. Lett. 2018, 23, 26; and Yang et al., J Clin Oncol. 2014, 44 (2): 159-67. It is also overly expressed in various solid tumors, including bladder urothelial carcinoma, breast invasive, colon adenocarcinoma, hepatocellular carcinoma, lung adenocarcinoma, pancreatic adenocarcinoma, rectum adenocarcinoma, stomach adenocarcinoma, thyroid carcinoma. Chen et al., Cell Oncol. 2020, 43:877-88.

A number of reports have suggested that HIF-1A either actives or inhibits metabolic disease. Gonzalez et al., Nat. Rev. Endocrinol. 2018, 15, 21-32; and Halberg et al., Mol Cell Biol. 2009, 16:4467-83. Obesity causes a chronic hypoxic state in adipose tissue and the small intestine, which promotes HIF-1A signaling, resulting in adverse metabolic effects, including insulin resistance and non-alcoholic fatty liver disease accompanied by liver fibrosis. Norouzirad et al., Oxid Med Cell Longev. 2017, 2017:5350267. Inhibition of hypoxia signaling via overexpression of von Hippel-Lindau protein, an E3 ubiquitin ligase, or silencing HIF-1A can significantly reduce liver fibrosis induced by both CC14 and bile duct ligation. Wang et al., Sci Rep. 2017, 7:41038.

Described herein are interfering RNA molecules targeting an mRNA of human HIF1a (anti-HIF1a interfering RNA). Such anti-HIF1a interfering RNAs can suppress HIF1a expression via the RNA interference process, thereby benefiting treatment of diseases associated with HIF1a, for example, those discussed herein. As used herein, the term “interfering RNA” refers to any RNA molecule that can be used in inhibiting a target gene, including both mature RNA molecules that are directly involved in RNA interference (e.g., the 21-23 nt dsRNA disclosed herein) or a precursor molecule that produces the mature RNA molecule.

An anti-HIF1a interfering RNA comprises a fragment that is complementary (completely or partially) to a target site within the HIF1a mRNA. The fragment may be 100% complementary to the target site. Alternatively, the fragment may be partially complementary, e.g., including one or more mismatches or gaps but sufficient to form double-strand at the target site to mediate RNA interference.

In some embodiments, an interfering RNA disclosed herein targets a HIF1a mRNA site having one of the nucleotide sequences:

(1)
(SEQ ID NO: 11)
AUGAAGUGUACCCUAACUA;
(2)
(SEQ ID NO: 12)
AAGUCUGCAACAUGGAAGGUA;
(3)
(SEQ ID NO: 1)
AGGCCACAUUCACGUAUAU;
(4)
(SEQ ID NO: 13)
GGCCACAUUCACGUAUAUG;
(5)
(SEQ ID NO: 2)
UGAGGAAGUACCAUUAUAU;
(6)
(SEQ ID NO: 14)
AAGUUCACCUGAGCCUAAUAG;
(7)
(SEQ ID NO: 15)
ACUUUCUUGGAAACGUGUAA;
(8)
(SEQ ID NO: 3)
CCGGUUGAAUCUUCAGAUA;
(9)
(SEQ ID NO: 4)
GCGCAAGUCCUCAAAGCAC;
(10)
(SEQ ID NO: 16)
GUCGGACAGCCUCACCAAA;
and
(11)
(SEQ ID NO: 17)
AGCGCAAGUCCUCAAAGCAC.

In some examples, the anti-HIF1a interfering RNA disclosed herein target the HIF1a mRNA site having the nucleotide sequence of AGGCCACAUUCACGUAUAU (SEQ ID NO: 1). In some examples, the anti-HIF1a interfering RNA disclosed herein target the HIF1a mRNA site having the nucleotide sequence of UGAGGAAGUACCAUUAUAU (SEQ ID NO: 2). In other examples, the anti-HIF1a interfering RNA disclosed herein target the HIF1a mRNA site having the nucleotide sequence of CCGGUUGAAUCUUCAGAUA (SEQ ID NO: 3). Exemplary anti-HIF1a interfering RNAs are provided in Tables 3 and 4 below.

In some embodiments the interfering RNA discloses herein may be a siRNA, i.e., a double-strand RNA (dsRNA) that contains two separate and complementary RNA chains. Such an siRNA may comprise a sense chain having a nucleotide sequence corresponding to the target HIF1a mRNA site and an antisense chain complementary to the sense chain (and the target site). It would have been known to those skilled in the art that the sense chain and/or the antisense chain does not need to be completely the same or complementary to the target site. One or more mismatches would be allowed as long as the siRNA can still target the mRNA site via base-pairing to mediate the RNA interference process. In some instances, the sense chain and/or the antisense chain (whole or a portion thereof) is completely the same or complementary to the target site. Exemplary siRNAs targeting HIF1a can be found in Tables 3 and 4 below.

In other examples, the interfering RNA discloses here can be a short hairpin RNA (shRNA), which is a RNA molecule forming a tight hairpin structure. Both siRNAs and shRNAs can be designed based on the sequence of the target mRNA sites of HIF1a as disclosed herein.

In some embodiments, the anti-HIF1a interfering RNAs disclosed herein can be an siRNA molecule, for example, those listed in Table 3 and Table 4 below. In specific examples, the siRNA is one of those listed in Table 4, for example, AI3-UM4 and AT9-UM4.

In some examples, the siRNA may comprise a sense chain comprising 5′-AGGCCACAUUCACGUAUAA-3′ (SEQ ID NO: 7) and an antisense chain comprising 5′-UUAUACGUGAAUGUGGCCUGU-3′ (SEQ ID NO: 8), e.g., A13-UM4. In some examples, the siRNA may comprise a sense chain comprising 5′-UGAGGAAGUACCAUUAUAA-3′ (SEQ ID NO: 9) and an antisense chain comprising 5′-UUAUAAUGGUACUUCCUCAAU-3′ (SEQ ID NO: 10), e.g., AT9-UM4.

In some instances, the siRNA disclosed herein may comprise the same sense chain and/or same antisense chain as AI3-UM4 or AT9-UM4. In other instances, the siRNA disclosed herein may comprise a sense chain that is at least 80% (e.g., at least 85%, at least 90%, at least 95%, or higher) identical to the sense chain of AI3-UM4 and/or comprise an antisense chain that is at least 80% (e.g., at least 85%, at least 90%, at least 95%, or higher) identical to the antisense chain of AI3-UM4. In other instances, the siRNA disclosed herein may comprise a sense chain that is at least 80% (e.g., at least 85%, at least 90%, at least 95%, or higher) identical to the sense chain of AT9-UM4 and/or comprise an antisense chain that is at least 80% (e.g., at least 85%, at least 90%, at least 95%, or higher) identical to the antisense chain of AT9-UM4.

The “percent identity” of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

In other embodiments, the anti-HIF1a siRNA described herein may contain up to 6 (e.g., up to 6, 5, 4, 3, or 2) nucleotide variations as compared with the sense chain and antisense chain (collectively or separately) of a reference siRNA, such as those listed in Table 3 or Table 4, for example, AI3-UM4 or AT9-UM4.

In some embodiments, any of the anti-HIF1a interfering RNAs (e.g., siRNAs such as AI3-UM4 or AT9-UM4) described herein may contain non-naturally-occurring nucleobases, sugars, or covalent internucleoside linkages (backbones). Such a modified oligonucleotide confers desirable properties, for example, enhanced cellular uptake, improved affinity to the target nucleic acid, increased in vivo stability, enhance in vivo stability (e.g., resistant to nuclease degradation), and/or reduce immunogenicity.

In one example, the anti-HIF1a interfering RNAs (e.g., siRNAs such as AI3-UM4 or AT9-UM4) described herein has a modified backbone, including those that retain a phosphorus atom (see, e.g., U.S. Pat. Nos. 3,687,808:4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In some examples, the anti-HIF1α interfering RNAs (e.g., siRNAs such as AI3-UM4 or AT9-UM4) described herein may contain the PS internucleotide linkages at the positions disclosed herein (e.g., Positions 5-8 within the seed region, such as at Positions between 5 and 6 and/or between 6 and 7). Alternatively or in addition, the anti-HIF1a interfering RNAs (e.g., siRNAs such as AI3-UM4 or AT9-UM4) described herein may also contain one or more additional modifications such as modified sugar, modified base, modified nucleotide, etc. including those disclosed herein. The anti-HIF1a interfering RNAs may also be conjugated to a targeting moiety, e.g., those disclosed herein. For example, the anti-HIF1a interfering RNA may be conjugated to a ligand (targeting moiety) or encapsulated into vesicles that can facilitate the delivery of siRNA to desired cells/tissues and/or facilitate cellular uptake. Suitable ligands include, but are not limited to, carbohydrate, peptide, antibody, polymer, small molecule and cholesterol. For example, one or more GalNAc moieties (e.g., a tri-GalNAc moiety) may be used as the targeting moiety for delivering the anti-HIF1a interfering RNA to liver cells.

Unless explicitly pointed out (e.g., PS bonds indicated by the symbol ‘*’), the unmodified nucleotide sequences provided herein are meant to encompass both unmodified RNA molecules and RNA molecules having any suitable modifications.

Any of the anti-HIF1a interfering RNA molecules (as well as the modified siRNA molecules) described herein can be prepared by conventional methods, e.g., chemical synthesis or in vitro transcription. Their intended bioactivity as described herein can be verified by, e.g., those described in the Examples below. In some instances, the modified siRNA molecule or the anti-HIF1a interfering RNA disclosed herein is capable of suppressing the expression of the target gene by at least 50%, e.g., by at least 65%, by at least 75%, by at least 80%, by at least 85%, by at least 90%, by at least 95%, or above.

Vectors for expressing any of the anti-HIF1a interfering RNAs are also within the scope of the present disclosure. The expression vector can comprise control elements (promoter/enhancers) operably linked to sequences coding for the anti-HIF1a interfering RNAs. Typically, these sequences are capable of coding of both the sense and the antisense strands of the anti-HIF1a interfering RNAs.

### III. Pharmaceutical Compositions

Any of the modified siRNA molecule or anti-HIF1a interfering RNAs as disclosed herein may be formulated into a suitable pharmaceutical composition. The pharmaceutical compositions as described herein can further comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride: phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (nonionic surfactants), or polyethylene glycol (PEG).

In some examples, the pharmaceutical composition described herein includes excipients that may include, but not limited to, trichloromono-fluoromethane, dichloro-difluoromethane, dichloro-tetrafluoroethane, chloropenta-fluoroethane, monochloro-difluoroethane, difluoroethane, tetrafluoroethane, heptafluoropropane, octafluoro-cyclobutane, purified water, ethanol, propylene glycol, glycerin, PEG (e.g., PEG400, PEG 600, PEG 800 and PEG 1000), sorbitan trioleate, soya lecithin, lecithin, oleic acid, Polysorbate 80, magnesium stearate and sodium laury sulfate, methylparaben, propylparaben, chlorobutanol, benzalkonium chloride, cetylpyridinium chloride, thymol, ascorbic acid, sodium bisulfite, sodium metabisulfite, EDTA, sodium hydroxide, tromethamine, ammonia, HCl, H₂SO₄, HNO₃, citric acid, CaCl₂), CaCO₃, sodium citrate, sodium chloride, disodium EDTA, saccharin, menthol, ascorbic acid, glycine, lysine, gelatin, povidone K25, silicon dioxide, titanium dioxide, zinc oxide, lactose, lactose monohydrate, lactose anhydrate, mannitol, and dextrose.

In other examples, the pharmaceutical composition described herein can be formulated in sustained-release format. Suitable examples of sustained-release preparations include semipermeable matrices of solid hydrophobic polymers which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic compositions are generally placed into a container having a sterile access port, for example, an intravenous solution bag or vial having a stopper pierceable by a hypodermic injection needle or a sealed container to be manually accessed.

The pharmaceutical compositions described herein can be in unit dosage forms such as solids, solutions or suspensions, or suppositories, for administration by inhalation or insufflation, intrathecal, intrapulmonary or intracerebral routes, oral, parenteral or rectal administration.

For preparing solid compositions, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as powder collections, tablets, pills and capsules. This solid preformulation composition is then subdivided into unit dosage forms of the type described above containing a suitable amount of the active ingredient in the composition.

Suitable surface-active agents include, in particular, non-ionic agents, such as polyoxyethylenesorbitans (e.g., TWEEN® 20, 40, 60, 80 or 85) and other sorbitans (e.g., SPAN® 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID™, LIPOSYN™, INFONUTROL™, LIPOFUNDIN™, and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. In some embodiments, the compositions are composed of particle sized between 10 nm to 100 mm.

Compositions in preferably sterile pharmaceutically acceptable solvents may be nebulized by use of gases. Nebulized solutions may be breathed directly from the nebulizing device or the nebulizing device may be attached to a face mask, tent, endotracheal tube and/or intermittent positive pressure breathing machine (ventilator). Solution, suspension or powder compositions may be administered, preferably orally or nasally, from devices which deliver the formulation in an appropriate manner.

In some embodiments, any of the modified siRNA molecule or anti-HIF1a interfering RNAs can be encapsulated or attached to a liposome, which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, any of the modified siRNA molecule or anti-HIF1α interfering RNAs may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

Any of the pharmaceutical compositions comprising the modified siRNA molecule disclosed herein may further comprise a component that enhances transport of the composition from endosomes and/or lysosomes to cytoplasm. Examples include a pH-sensitive agent (e.g., a pH-sensitive peptide).

In some embodiments, any of the pharmaceutical compositions herein may further comprise a second therapeutic agent based on the intended therapeutic uses of the composition.

### IV. Suppressing Target Gene Expression

Any of the modified siRNA molecules or anti-HIF1a interfering RNA molecules disclosed herein may be used to suppress expression of the target gene (e.g., HIF1a) either in vivo or in vitro.

To practice the method disclosed herein, an effective amount of the pharmaceutical composition described herein that comprise the modified siRNA molecule can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, intratumoral, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution.

As used herein, “an effective amount” refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of the modified siRNAs or anti-HIF1a interfering RNAs may be appropriate. Various formulations and devices for achieving sustained release are known in the art.

Generally, for administration of any of the modified siRNA molecules or any of the anti-HIF1a interfering RNAs described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the siRNAs, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen can vary over time.

In some embodiments, for an adult patient of normal weight, doses ranging from about 0.3 to 5.00 mg/kg may be administered. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of the modified siRNA or the anti-HIF1a interfering RNA molecule as described herein will depend on the type and severity of the disease/disorder, whether the modified siRNA or anti-HIF1a interfering RNA is administered for preventive or therapeutic purposes, previous therapy, the patient's clinical history and response to the antagonist, and the discretion of the attending physician. A clinician may administer the modified siRNA molecule or the anti-HIF1a interfering RNA until a dosage is reached that achieves the desired result. In some embodiments, the desired result is a decrease in tumor burden, a decrease in cancer cells, or increased immune activity.

Methods of determining whether a dosage resulted in the desired result would be evident to one of skill in the art. Administration of one or more modified siRNA molecule or the anti-HIF1a interfering RNA can be continuous or intermittent, depending, for example, upon the recipient's physiological condition, whether the purpose of the administration is therapeutic or prophylactic, and other factors known to skilled practitioners. The administration of the modified siRNA molecule or the anti-HIF1a interfering RNA may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intratumoral, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some embodiments, the composition can be administered via a nasal route, for example, intranasal spray, nasal spray, or nasal drops.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, the modified siRNAs can be administered by the drip method, whereby a pharmaceutical formulation containing the interfering RNA and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the modified siRNAs disclosed herein, can be dissolved and administered in a pharmaceutical excipient such as Water-for-Injection, 0.9% saline, or 5% glucose solution.

In one embodiment, the modified siRNA molecule is administered via site-specific or targeted local delivery techniques. Examples of site-specific or targeted local delivery techniques include various implantable depot sources of the therapeutic RNA molecule or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing a polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

In some embodiments, any of the modified siRNA molecule, any of the anti-HIF1α interfering RNA, or a pharmaceutical composition comprising such can be administered by pulmonary delivery system, that is, the active pharmaceutical ingredient is administered into lung. The pulmonary delivery system can be an inhaler system. In some embodiment, the inhaler system is a pressurized metered dose inhaler, a dry powder inhaler, or a nebulizer. In some embodiment, the inhaler system is with a spacer.

In some embodiment, the pressurized metered dose inhaler includes a propellent, a co-solvent, and/or a surfactant. In some embodiment, the propellent is selected from the group comprising of fluorinated hydrocarbons such as trichloromono-fluoromethane, dichloro-difluoromethane, dichloro-tetrafluoroethane, chloropenta-fluoroethane, monochloro-difluoroethane, difluoroethane, tetrafluoroethane, heptafluoropropane, octafluoro-cyclobutane. In some embodiment, the co-solvent is selected from the group comprising of purified water, ethanol, propylene glycol, glycerin, PEG400, PEG 600, PEG 800 and PEG 1000. In some embodiment, the surfactant or lubricants is selected from the group comprising of sorbitan trioleate, soya lecithin, lecithin, oleic acid, Polysorbate 80, magnesium stearate and sodium laury sulfate. In some embodiment, the preservatives or antioxidants is selected from the group comprising of methyparaben, propyparaben, chlorobutanol, benzalkonium chloride, cetylpyridinium chloride, thymol, ascorbic acid, sodium bisulfite, sodium metabisulfite, sodium bisulfate, EDTA. In some embodiment, the pH adjustments or tonicity adjustments is selected from the group comprising of sodium oxide, tromethamine, ammonia, HCl, H₂SO₄, HNO₃, citric acid, CaCl₂), CaCO₃.

In some embodiment, the dry powder inhaler includes a disperse agent. In some embodiment, the disperse agent or carrier particle is selected from the group comprising of lactose, lactose monohydrate, lactose anhydrate, mannitol, dextrose which their particle size is about 1-100 μm.

In some embodiment, the nebulizer may include a co-solvent, a surfactant, lubricant, preservative and/or antioxidant. In some embodiment, the co-solvent is selected from the group comprising of purified water, ethanol, propylene glycol, glycerin, PEG (e.g., PEG400, PEG600, PEG800 and/or PEG 1000). In some examples, the surfactant or lubricant is selected from the group comprising of sorbitan trioleate, soya lecithin, lecithin, oleic acid, magnesium stearate and sodium laury sulfate. In some examples, the preservative or antioxidant is selected from the group comprising of methyparaben, propyparaben, chlorobutanol, benzalkonium chloride, cetylpyridinium chloride, thymol, ascorbic acid, sodium bisulfite, sodium metabisulfite, sodium bisulfate, EDTA. In some examples, the nebulizer further includes a pH adjustment or a tonicity adjustment, which is selected from the group comprising of sodium oxide, tromethamine, ammonia, HCl, H₂SO₄, HNO₃, citric acid, CaCl₂), CaCO₃.

In some embodiments, a DNA molecule capable of producing an anti-HIF1a interfering RNA or a pharmaceutical composition comprising such may be used for silencing HIF1a expression. A pharmaceutical composition comprising such a DNA molecule (e.g., a vector) may be administered to a subject in need of the treatment in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The term “about” or “approximately” used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

A subject to be treated by any of the modified siRNA molecules or the anti-HIF1a interfering RNAs may have or suspected of having a disease associated with the target gene, suppressing of which can be achieved by the modified siRNA molecules (see Target Genes disclosed above) or the anti-HIF1a interfering RNA (HIF1a). The terms “subject,” “individual,” and “patient” are used interchangeably herein and refer to a mammal being assessed for treatment and/or being treated. Subjects may be human, but also include other mammals, particularly those mammals useful as laboratory models for human disease, e.g. mouse, rat, rabbit, dog, monkey etc. A human subject who needs the treatment may be a human patient having, at risk for, or suspected of having a target disease/disorder, such as tumor.

Any of the modified siRNA molecules disclosed herein may be used for treating a disease or disorder associated with the target gene. Exemplary diseases include, but are not limited to, cancer, fibrosis, a metabolic disease, a cardiovascular disease, an immune disease, or an inheritance disorder.

Any of the anti-HIF1a interfering RNAs as disclosed herein may be used for treating a disease or disorder associated with HIF1a. Examples include, but are not limited to, solid tumors, cancers, ischemic heart disease, congestive heart failure, acute lung injury, pulmonary hypertension, pulmonary fibrosis, chronic obstructive pulmonary disease, acute liver failure, liver fibrosis and cirrhosis, acute kidney injury, chronic kidney disease, obesity and diabetes mellitus.

Any of the modified siRNAs or anti-HIF1a interfering RNAs disclosed herein may be used in a combined therapy with one or more additional therapeutic agents for treating the target disease. The term combination therapy, as used herein, embraces administration of these agents (e.g., the modified siRNA molecule, the anti-HIF1a interfering RNA and the additional therapeutic agents) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route including, but not limited to, oral routes, intravenous routes, intramuscular, intratumoral, subcutaneous routes, direct absorption through mucous membrane tissues, and pulmonary delivery routes. The agents can be administered by the same route or by different routes. For example, a first agent can be administered by pulmonary delivery routes, and a second agent can be administered intravenously.

As used herein, the term “sequential” means, unless otherwise specified, characterized by a regular sequence or order, e.g., if a dosage regimen includes the administration of a composition and an antiviral agent, a sequential dosage regimen could include administration of the composition before, simultaneously, substantially simultaneously, or after administration of the antiviral agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the agents described herein.

Combination therapy can also embrace the administration of the agents described herein, in further combination with other biologically active ingredients and non-drug therapies. It should be appreciated that any combination of a composition described herein and a second therapeutic agent may be used in any sequence for treating a target disease.

Treatment efficacy for a target disease/disorder can be assessed by methods well-known in the art.

In some embodiments, any of the modified siRNAs or anti-HIF1α interfering RNAs may be used to suppress expression of the target gene in vitro. To perform such a method, the modified siRNA or the anti-HIF1a interfering RNA (e.g., via an encoding nucleic acid such as a vector) may be in contact with cells cultured in vitro, e.g., for research purposes such as for studying disease mechanisms and/or for drug candidate validation.

### V. Kits

The present disclosure can be used alone or as a component of a kit having at least one of the reagents necessary to carry out the in vitro or in vivo introduction of siRNA to test samples and/or subjects.

For example, preferred components of the kit include the modified siRNA molecule of the disclosure and a vehicle that promotes introduction of the siRNA into cells of interest as described herein (e.g., using lipids and other methods of transfection known in the art, see for example Beigelman et al, U.S. Pat. No. 6,395,713).

The kit can also be used for target validation, such as in determining gene function and/or activity, or in drug optimization, and in drug discovery (see for example Usman et al., U.S. Ser. No. 60/402,996). Such a kit can also include instructions to allow a user of the kit to practice the disclosure. Such kits can optionally include one or more of the second therapeutic agents as also described herein.

In some embodiments, the kit can comprise instructions for use in accordance with any of the methods described herein. The kit may further comprise a description of selecting an individual suitable for treatment based on identifying whether that individual has the disease or is at risk for the disease.

The instructions relating to the use of the modified siRNA molecule to achieve the intended therapeutic effects generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk, or QR code) are also acceptable.

The label or package insert may indicate that the composition is used for the intended therapeutic utilities. Instructions may be provided for practicing any of the methods described herein.

The kits of this disclosure are in suitable packaging. Suitable packaging includes, but is not limited to, chambers, vials, bottles, jars, flexible packaging (e.g., sealed Mylar or plastic bags), and the like. Also contemplated are packages for use in combination with a specific device, such as an inhaler, nebulizer, ventilator, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container. In some embodiments, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Investigation of Off-Target Effects of Modified siRNA Molecules

Seven siRNA candidates listed in Table 1 were synthesized and screened for the assessment of transcriptome-wide off-target effects. In addition to PS linkages introduced at the 5′ and/or 3′ terminals of the antisense strand to resist enzymatic degradation, PS linkage was also incorporated in the seed region of the antisense strand as shown in Table 1.

TABLE 1
Sequences of HIF1A siRNA with PS linkage
targeting human HIF1A mRNA

| | Sequence of | SEQ | Sequence of | SEQ |
|---|---|---|---|---|
| SiRNA | sense strand | ID | antisense | ID |
| Candidates | (5′-3′) | NO: | strand (5′-3′) | NO: |
| AI3-A-PS1 | CCACAUUCA | 18 | U*U*AUACGUGAA | 19 |
| | CGUAUAA | | UGUGGCCU*G*U | |
| AI3-A-PS2 | CCACAUUCA | 18 | U*U*AUACG*UGA | 20 |
| | CGUAUAA | | AUGUGGCCU*G*U | |
| AI3-A-PS3 | CCACAUUCA | 18 | U*U*AUAC*GUGA | 21 |
| | CGUAUAA | | AUGUGGCCU*G*U | |
| AI3-A-PS4 | CCACAUUCA | 18 | U*U*AUA*CGUGA | 22 |
| | CGUAUAA | | AUGUGGCCU*G*U | |
| AI3-A-PS5 | CCACAUUCA | 18 | U*U*AUAC*G*UG | 23 |
| | CGUAUAA | | AAUGUGGCCU*G*U | |
| AI3-A-PS6 | CCACAUUC | 18 | U*U*AUA*C*GUGA | 24 |
| | ACGUAUAA | | AUGUGGCCU*G*U | |
| AI3-A-PS7 | CCACAUUC | 18 | U*U*AUA*C*G*UG | 25 |
| | ACGUAUAA | | AAUGUGGCCU*G*U | |

*represent phosphorothioate (PS) bond

The human hepatocyte HL-7702 cells were cultured in RPMI-1640 medium supplemented with 10% fetal bovine serum. The human hepatocellular carcinoma (HepG2) cells were cultured in minimum essential medium (Gibco, ThermoFisher Scientific) with 10% fetal bovine serum (Gibco, ThermoFisher Scientific).

Briefly, the siRNA candidates listed in Table 1 were transfected into human hepatocellular carcinoma HepG2 cell line or human hepatocyte HL-7702 cell line to compare off-target effects caused by these siRNAs. HepG2 cells were seeded into 24-well culture plates and transfected with an siRNA candidate for 24 hr. After 24 hr transfection, total RNAs were isolated from the siRNA-transfected cells to evaluate the knockdown efficiencies of HIF1A mRNA using RT-qPCR.

For off-target analysis, the HL-7702 cells were seeded into 6-well culture plates and transfected with the siRNA for 24 hr. Total RNA was then isolated and genome-wide RNA sequencing was performed to assess transcriptome-wide off-target effects.

For the RNA-seq experiment, the HL-7702 cells were seeded at 2×10⁵ cells/well in 6-well culture plates and incubated for 18 hr. Each siRNA candidate (10 nM) was then transfected into the HL-7702 cells using Lipofectamine RNAiMAX (9 ul/well; Thermo Fisher Scientific) following the manufacturer's protocol. After 24-hr transfection, cells were washed twice with 1× dPBS and solubilized in TRIzol reagent (Thermo Fisher Scientific). Total RNA was extracted following the manufacturer's instructions. Total RNA was extracted and treated with DNase to avoid genomic DNA contamination.

Purity (A260/A280 and A260/A230 ratio) and quality (RIN≥8.0) of the extracted RNA were determined using a NanoDrop 2000 spectrophotometer (Thermo Scientific) and an Agilent bioanalyzer 2100 (Agilent Technologies, Santa Clara, CA). Quality of all extracted RNA samples was A260/A280≥1.9, A260/A230≥2, and RIN=10.0. RNA-seq Libraries was prepared using Truseq Stranded Total RNA Library Prep Gold (Illumina) and sequenced on the NovaSeq 6000 sequencer (Illumina) according to manufacturers' instructions. Average of 84.5 million reads per sample was obtained from 2×150-bp paired-end sequencing. Raw RNA reads were filtered with minimal mean quality scores of 20 using SeqPrep and Sickle. Filtered reads were aligned to the human genome (GRCh.38.p13) using HISAT2 and then assembled using StringTie. The gene expression level was qualified by RSEM and normalized by the fragments per kilobase per million mapped reads (FPKM). Differential gene expression analysis was performed by DEGseq. The genes with false discovery rate (FDR)≤0.001 and fold change ≥2 were identified as differentially expressed genes (DEGs).

Genome-wide RNA sequencing, comparing HIF1A siRNA-treated with no-siRNA-treated (untreated) cells, was performed to determine whether PS linkage at positions 5-8 of the siRNA antisense strand had any major effect on the off-target events.

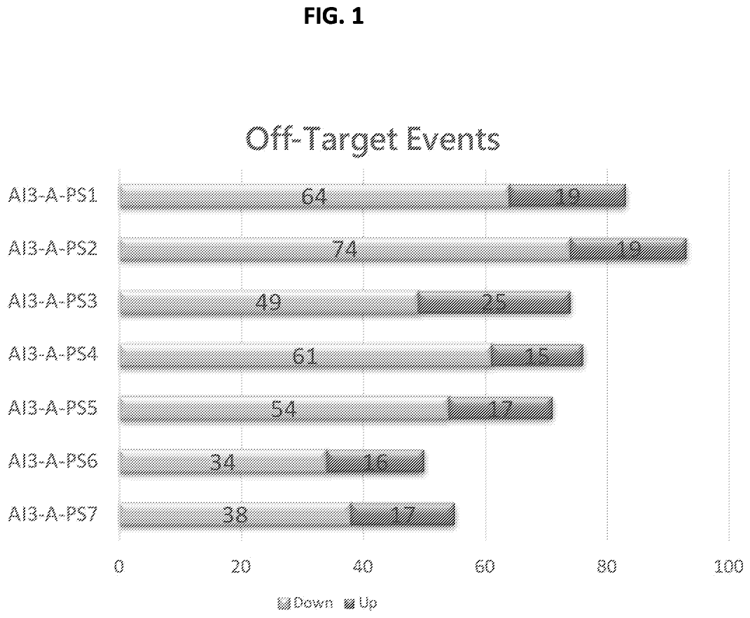

As shown in FIG. 1, 34˜74 down-regulated genes were observed between treated and untreated cells. The number of down-regulated genes caused by AI3-A-PS6 (34 genes) was reduced by 47% compared with that caused by AI3-A-PS1 (64 genes), which contained no PS linkage at position 5-8 of the antisense strand.

### Example 2: Investigation of Target Gene Knockdown Efficiency by RT-qPCR

The knockdown efficiency of siRNA candidates listed in Table 1 were determined by RT-qPCR. Briefly, the HepG2 cells were seeded at 5×10⁵ cells/well in 24-well culture plates. Each siRNA candidate (10 nM) was then transfected into HepG2 cells using Lipofectamine RNAiMAX (3 ul/well). After 24-hr transfection, total RNA was isolated using an RNeasy kit (Qiagen) according to the manufacturer's protocol.

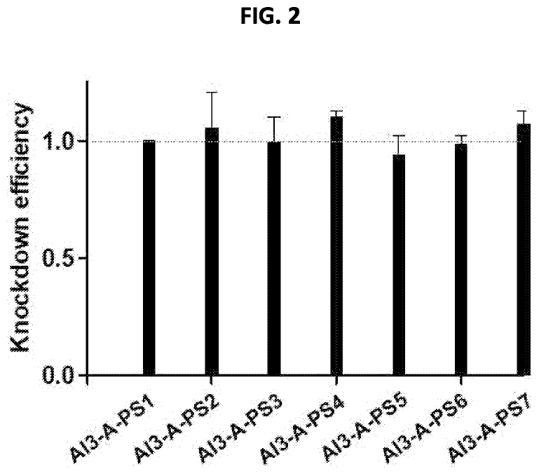

HIF1A mRNA levels was quantified using one-step real-time quantitative PCR with iTaq Universal Probes one-step kit (Bio-Rad), performed on the LightCycler 480 (Roche Diagnostics). Primers and Probes for HIF1A were from predesigned PrimeTime qPCR assays (Integrated DNA Technologies). Each sample was assayed in triplicate to determine an average threshold cycle (Ct) value. Gene expression fold change was calculated using the ΔΔCt method. HIF1A mRNA was normalized to constitutively expressed GAPDH mRNA, as depicted in FIG. 2.

As shown in FIG. 2, the knockdown efficiencies of siRNA AI3-A-PS2 to siRNA AI3-A-PS7 were similar after normalizing with siRNA AI3-A-PS1-treated cells. The PS linkage at position 5-8 of the siRNA antisense strand does not affect the knockdown efficiency of the siRNA.

### Example 3: Development of Anti-HIF1A siRNAs with High Knockdown Efficiency

This example reports the identification of anti-HIF1A siRNAs with high efficiency in interfering HIF1A expression.

Design of Candidate Anti-HIF1A siRNAs

siRNA candidates targeting human HIF1A mRNA sequence (GenBank No. NM_001530.4) (anti-HIF1A siRNAs) were designed and those with low off-target possibility were then selected based on the following: (1) low cross-reactivity to human mRNA database; and (2) low number of essential genes predicted to be targeted by the siRNA candidates. A first set of siRNAs was synthesized and formed into duplexes as shown in Table 3 below. The first set of HIF1A siRNA (Table 3 below) were screened for HIF1A mRNA suppression in HepG2 cells. The HepG2 cells were transfected with these siRNA for 24 hr. HIF1A expression were then measured using real-time qPCR.

### Culture of HepG2 Cells

The human hepatocellular carcinoma (HepG2) cell line was cultured in minimum essential medium (Gibco, ThermoFisher Scientific, USA) containing 10% fetal bovine serum (Gibco, ThermoFisher Scientific, USA), 200 units/mL penicillin plus 200 units/mL streptomycin at 37° C. with 5% CO2.

siRNA Transfection

HepG2 cells were seeded at 5×10⁵ cells/well in 24-well culture plates. After 18 hr incubation, the medium was replaced with 500 ul of fresh growth medium. The complex composition of siRNA and RNAiMax for each well was prepared as following: (1) 1 ul of siRNA was added to 50 ul of Opti-MEM; (2) 1.5 ul of RNAiMax was added to 50 ul of Opti-MEM; (3) Gently mix (1) and (2) and incubate at room temperature for 10 minutes. Transfection was carried out by adding 100 ul of siRNA/RNAiMax complex to each well. Cells were then incubated for 24 hr prior to RNA purification.

### RT-qPCR

Total RNA was isolated using an RNeasy kit (Qiagen) according to the manufacturer's protocol. HIF1A mRNA levels was quantified using one-step real-time quantitative PCR with iTaq Universal Probes one-step kit (Bio-Rad), performed on the LightCycler 480 (Roche Diagnostics). RT-qPCR was performed in triplicates with 50 ng of total RNA, 500 nM each of forward, reverse primer and probe, 0.25 ul of reverse transcriptase, and 2×iTaq Master Mix in a total volume of 10 ul in a 384-well plate. Primers and Probes for HIF1A and GAPDH (Table 2) were synthesized from Integrated DNA Technologies. The cycling condition was in accordance with the manufacturer's recommended cycling parameters: 50° C. for 10 min, 95° C. for 2 min, and 40 cycles of 95° C. for 15 s and 60° C. for 1 min. Gene expression fold change was calculated using the ΔΔCt method. HIF1A mRNA was normalized to constitutively expressed GAPDH mRNA.

TABLE 2
Primers and probes used in RT-qPCR

| | | | SEQ |
|---|---|---|---|
| | Primer/ | | ID |
| Gene | Probe | Sequence | NO: |
| HIF1A | HIF1A_F | 5′-CTCTGATCATCTGA | 26 |
| | | CCAAAACTCA-3′ | |
| | HIF1A_R | 5′-CAACCCAGACATATCCACCTC-3′ | 27 |
| | HIF1A_ | 5′-/56FAM/TGGCAAGCA/ZEN/ | 28 |
| | Probe | TCCTGTACTGTCCTG/3IABkFQ/-3′ | |
| GAPDH | GAPDH_F | 5′-ACATCGCTCAGACACCATG-3′ | 29 |
| | GAPDH_R | 5′-TGTAGTTGAGGTCAATGAAGGG-3′ | 30 |
| | GAPDH_ | 5′-56FAM/AAGGTCGGA/ZEN/ | 31 |
| | Probe | GTCAACGGATTTGGTC/ | |
| | | 3IABkFQ/-3′ | |

### Result

In the first round of screening, HepG2 cells were treated with 30 nM HIF1A siRNA. The results are shown in Table 3. The relative expression rate of HIF1A mRNA in HepG2 cells treated with siRNA are expressed as % HIF1A mRNA relative to control treated with RNAiMax only and no siRNA.

TABLE 3
Relative Expression Rates of HIF1A mRNA
in Human Liver Cancer Cells Treated
with siRNA

| | | | | | HIF-1A |
|---|---|---|---|---|---|
| | | | | | mRNA |
| | | SEQ | | SEQ | |
| Duplex | Sense | ID | Antisense | ID | expression |
| no | (5′-3′) | NO: | (5′-3′) | NO: | rate (%) |
| 1 | AAGUGUACCC | 32 | UAGUUAGGGU | 43 | 34.6 |
| | UAACUA | | ACACUUCAU | | |
| 2 | UGCAACAUGG | 33 | UACCUUCCAU | 44 | 38.2 |
| | AAGGUA | | GUUGCAGACU | | |
| | | | U | | |
| 3 | AGGCCACAUU | 1 | AUAUACGUGA | 45 | 4.4 |
| | CACGUAUAU | | AUGUGGCCUd | | |
| | | | TdT | | |
| 4 | CCACAUUCAC | 34 | AUAUACGUGA | 45 | 5.0 |
| | GUAUAU | | AUGUGGCCUd | | |
| | | | TdT | | |
| 5 | CCACAUUCAC | 34 | AUAUACGUGA | 46 | 20.6 |
| | GUAUAU | | AUGUGGCCU | | |
| 6 | CACAUUCACG | 35 | CAUAUACGUG | 47 | 31.1 |
| | UAUAUG | | AAUGUGGCC | | |
| 7 | GGAAGUACCA | 36 | AUAUAAUGGU | 48 | 7.2 |
| | UUAUAU | | ACUUCCUCA | | |
| 8 | CACCUGAGCC | 37 | CUAUUAGGCU | 49 | 27.2 |
| | UAAUAG | | CAGGUGAACU | | |
| | | | U | | |
| 9 | UCUUGGAAAC | 38 | UUACACGUUU | 50 | 16.6 |
| | GUGUAA | | CCAAGAAAGU | | |
| 10 | GUUGAAUCUU | 39 | UAUCUGAAGA | 51 | 7.0 |
| | CAGAUA | | UUCAACCGGd | | |
| | | | TdT | | |
| 11 | CAAGUCCUCA | 40 | GUGCUUUGAG | 52 | 7.6 |
| | AAGCAC | | GACUUGCGCd | | |
| | | | TdT | | |
| 12 | GGACAGCCUC | 41 | UUUGGUGAGG | 53 | 34.4 |
| | ACCAAA | | CUGUCCGACd | | |
| | | | TdT | | |
| 13 | CAAGUCCUCA | 42 | GUGCUUUGAG | 54 | 12.3 |
| | AAGCACU | | GACUUGCGCU | | |
| | | | dTdT | | |

Duplex Nos. 3, 7 and 10 were further modified as listed in Table 4. Second round of screening was carried out in HepG2 cells treated with 1 nM siRNA. The siRNA transfection and RT-qPCR was performed as previously described. The HIF1A expression level caused by each siRNA duplex is presented in Table 4.

TABLE 4
Relative Expression Rates of HIF1A mRNA
in Human Liver Cancer Cells Treated
with Modified siRNA Candidates

| | | | | | HIF-1A |
|---|---|---|---|---|---|
| | | | | | mRNA |
| | | SEQ | | SEQ | expres- |
| | Sense | ID | Antisense | ID | sion |
| siRNA | (5′-3′) | NO: | (5′-3′) | NO: | rate (%) |
| AI3-UM1 | AGGCCACAUU | 55 | AUAUACGUGA | 46 | 30.8 |
| | CACGUAUAU | | AUGUGGCCU | | |
| AI3-UM2 | AGGCCACAUU | 55 | AUAUACGUGA | 59 | 33.3 |
| | CACGUAUAU | | AUGUGGCCUU | | |
| | | | U | | |
| AI3-UM3 | AGGCCACAUU | 7 | UUAUACGUGA | 60 | 37.3 |
| | CACGUAUAA | | AUGUGGCCUU | | |
| | | | U | | |
| AI3-UM4 | AGGCCACAUU | 7 | UUAUACGUGA | 8 | 29.9 |
| | CACGUAUAA | | AUGUGGCCUG | | |
| | | | U | | |
| AT9-UM1 | GGAAGUACCA | 56 | AUAUAAUGGU | 61 | 44.4 |
| | UUAUAU | | ACUUCCUCAU | | |
| | | | U | | |
| AT9-UM2 | GGAAGUACCA | 57 | UUAUAAUGGU | 62 | 55.9 |
| | UUAUAA | | ACUUCCUCAU | | |
| | | | U | | |
| AT9-UM3 | GGAAGUACCA | 57 | UUAUAAUGGU | 63 | 48.9 |
| | UUAUAA | | ACUUCCUCAU | | |
| | | | C | | |
| AT9-UM4 | UGAGGAAGUA | 9 | UUAUAAUGGU | 10 | 42.1 |
| | CCAUUAUAA | | ACUUCCUCAA | | |
| | | | U | | |
| AG23-UM1 | GUUGAAUCUU | 39 | UAUCUGAAGA | 64 | 45.8 |
| | CAGAUA | | UUCAACCGG | | |
| AG23-UM2 | GUUGAAUCUU | 39 | UAUCUGAAGA | 65 | 45.1 |
| | CAGAUA | | UUCAACCGGU | | |
| | | | U | | |
| AG23-UM3 | GUUGAAUCUU | 58 | AAUCUGAAGA | 66 | 40.7 |
| | CAGAUU | | UUCAACCGGU | | |
| | | | U | | |
| AG23-UM4 | GUUGAAUCUU | 58 | AAUCUGAAGA | 67 | 53.8 |
| | CAGAUU | | UUCAACCGGC | | |
| | | | G | | |

### Example 4: Effects of Anti-siRNAs in Human HepG2 Xenograft Mice

The knockdown effect of an exemplary anti-HIF1A siRNA (AI3-UM4, a.k.a., AI3) in an xenograft animal model was investigated as follows. HepG2 cells were inoculated subcutaneously into female M-NSG mice aged 4 weeks. When the tumors reached the average volume of 50 mm³, the mice were randomly divided into two groups according to tumor size (n=3 in each group) and then subcutaneously injected with PBS (vehicle) or HIF1A siRNA (10 mg/Kg) at dayl, d3, d7 and d14. AI3-UM4 (AI3) was used in this study as an example.

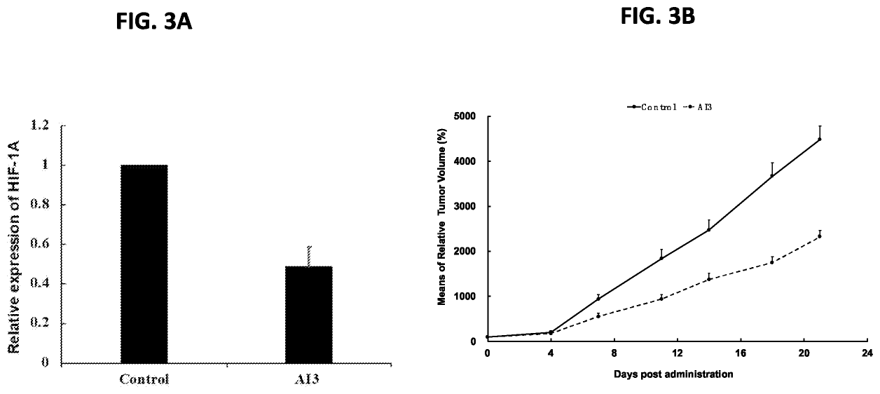

After twenty-one days, the mice were sacrificed and total RNAs were extracted from the tumor xenografts using a RNeasy kit (Qiagen) according to the manufacturer's protocol. The relative expression of HIF1A mRNA was quantified by RT-qPCR as previously described. The HIF1A expression level in human HepG2 tumor xenografts is presented in FIG. 3A. After normalized with control (treated with vehicle), HIF1A mRNA level treated with HIF1A siRNA (10 mg/Kg AI3) was reduced by 52% in human hepatocellular carcinoma cells.

Further, the anti-tumor growth effect of the exemplary anti-HIF1A siRNA was investigated in the xenograft mouse model. Female M-NSG mice bearing HepG2 tumors (˜50 mm³) were prepared and divided into two groups as described previously. Vehicle and HIF1A siRNA were injected subcutaneously to the mice at day 1, day 3, day 7 and dayl4. The length and width of the tumor in each mouse were measured twice a week for three weeks. Tumor volumes were calculated as L×W2×0.5. Relative tumor volume (%) is defined as the percentage of the tumor volume at each time point versus the initial tumor volume (at the initial time point of dosing) of each mouse. A shown in FIG. 3B, administration of HIF1A siRNA (10 mg/Kg AI3) significantly reduced tumor volume by 49% in human hepatocellular carcinoma cells.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A modified small interfering RNA (siRNA) molecule, comprising a sense strand and an antisense strand, wherein the antisense strand comprises phosphorothioate (PS) internucleotide linkages between nucleotides at positions 5 and 6 and/or between nucleotides at positions 6 and 7, and wherein the modified siRNA has reduced off-target effect as compared with the siRNA counterpart that has no PS internucleotide linkages between nucleotides at positions 5 and 6 and between nucleotides at positions 6 and 7.

2. The modified siRNA molecule of claim 1, wherein the antisense strand further comprises PS internucleotide linkages between nucleotides at positions 1 and 2 and/or between nucleotides at positions 2 and 3.

3. The modified siRNA molecule of claim 1, wherein the antisense strand is of 19-25 nucleotides in length.

4. The modified siRNA molecule of claim 1, wherein the antisense strand further comprises PS internucleotide linkages between the first and second nucleotides at the 3′ end and/or between the second and third nucleotides at the 3′ end.

5. The modified siRNA molecule of claim 3, wherein the antisense strand is of 21-nucleotides in length.

6. The modified siRNA of claim 5, wherein the antisense strand further comprises PS internucleotide linkages between the nucleotides at positions 19 and 20 and/or between nucleotides at positions 20 and 21.

7. The modified siRNA molecule of claim 1, wherein the modified siRNA molecule silences expression of a pathogenic gene, which optionally is a bacterial gene, a viral gene or a fungal gene.

8. The modified siRNA molecule of claim 1, wherein the modified siRNA molecule silences expression of a disease gene.

9. The modified siRNA molecule of claim 8, wherein the disease gene is involved in cancer, fibrosis, a metabolic disease, a cardiovascular disease, an immune disease, or an inheritance disorder.

10. The modified siRNA molecule of claim 9, wherein the disease gene is involved in cancer, optionally wherein the disease gene is selected from the group consisting of HIF1A, HIF2, IGF1R, VEGF, EREG, KRAS, ALK, BRAF, NRAS, STAT3, CDH2, KIFL1, PIK3CA, Src, RAS, RAF, and TP53.

11. The modified siRNA molecule of claim 9, wherein the disease gene is involved in fibrosis, optionally wherein the disease gene is selected from the group consisting of HIF1A, HIF1B, HIF2, TGF-β1, and CTGF.

12. The modified siRNA molecule of claim 9, wherein the disease gene is involved in a metabolic disease or a cardiovascular disease, optionally wherein the disease gene is selected from the group consisting of AGT, ApoC-III, and apoB.

13. The modified siRNA molecule of claim 9, wherein the disease gene is involved in an immune disease, optionally wherein the disease gene is selected from the group consisting of GATA-3, CCR3, TGF-β1, IL-6, TNF-α, IFN-γ, IL-1β, CCL2, and CCL10.

14. The modified siRNA molecule of claim 9, wherein the disease gene is involved in an inheritance disorder, optionally wherein the disease gene is apoB or PCSK9.

15. The modified siRNA molecule of claim 1, wherein the modified siRNA molecule is associated with a targeting moiety.

16. A pharmaceutical composition, comprising the modified siRNA molecule of claim 1 and a pharmaceutically acceptable carrier.

17. A method for silencing a target gene, comprising contacting the modified siRNA molecule of claim 1 or the pharmaceutical composition of claim 16 with cells expressing the target gene.

18. The method of claim 17, wherein the contacting step is performed by administering the modified siRNA molecule or the pharmaceutical composition to a subject in need thereof.

19. An interfering RNA that targets human hypoxia inducible factor 1 subunit alpha (HIF1α), wherein the interfering RNA comprises a nucleotide sequence complementary to a target site in a HIF1α mRNA, and wherein the target site in the HIF1α mRNA comprises the nucleotide sequence of:
(a)
(SEQ ID NO: 1)
AGGCCACAUUCACGUAUAU;
(b)
(SEQ ID NO: 2)
UGAGGAAGUACCAUUAUAU;
(c)
(SEQ ID NO: 3)
CCGGUUGAAUCUUCAGAUA;
(d)
(SEQ ID NO: 4)
GCGCAAGUCCUCAAAGCAC;
(e)
(SEQ ID NO: 5)
AGGCCACAUUCACGUAUA;
or
(f)
(SEQ ID NO: 6)
UGAGGAAGUACCAUUAUA

20. The interfering RNA of claim 19, wherein the interfering RNA is a small interfering RNA (siRNA) comprising a sense strand and an antisense strand.

21. The interfering RNA of claim 19, wherein the antisense strand is of 19-25 nucleotides in length.

22. The interfering RNA of claim 20, wherein the sense strand and the antisense strand comprises the following nucleotide sequences, respectively:
(a)
(SEQ ID NO: 7)
5′-AGGCCACAUUCACGUAUAA-3′
and
(SEQ ID NO: 8)
5′-UUAUACGUGAAUGUGGCCUGU-3′;
or
(b)
(SEQ ID NO: 9)
5′-UGAGGAAGUACCAUUAUAA-3′
and
(SEQ ID NO: 10)
5′-UUAUAAUGGUACUUCCUCAAU-3′

23. The interfering RNA of claim 20, wherein the antisense strand comprises phosphorothioate (PS) internucleotide linkages between nucleotides at Positions 5 and 6 and/or between nucleotides at Positions 6 and 7, and wherein the modified siRNA has reduced off-target effect as compared with the siRNA counterpart that has no PS internucleotide linkages between nucleotides at Positions 5 and 6 and between nucleotides at Positions 6 and 7.

24. The interfering RNA of claim 23, wherein the antisense strand further comprises PS internucleotide linkages between nucleotides at Positions 1 and 2 and/or between nucleotides at Positions 2 and 3.

25. The interfering RNA of claim 20, wherein the antisense strand further comprises PS internucleotide linkages between the first and second nucleotides at the 3′ end and/or between the second and third nucleotides at the 3′ end.

26. The interfering RNA of claim 19, wherein the interfering RNA comprises one or more modified nucleotides.

27. The interfering RNA of claim 19, wherein the one or more modified nucleotides comprise 2′-fluoro, 2′-O-methyl, or a combination thereof.

28. A pharmaceutical composition, comprising an interfering RNA targeting human HIF1α set forth in claim 19 and a pharmaceutically acceptable carrier.

29. A method for suppressing expression of human HIF1α, the method comprising contacting an effective amount of an interfering RNA set forth in claim 19 with a cell that expresses human HIF1α.

30. The method of claim 29, wherein the contacting step is performed by administering the effective amount of the interfering RNA or a pharmaceutical composition comprising such to a subject.

31. The method of claim 30, wherein the subject is a human patient having or suspected of having a disease associated with HIF1α.

32. The method of claim 31, wherein the disease associated with HIF1α is a cancer, a heart disease, a lung disease, a liver disease, a kidney disease, obesity, or diabetes.


SEQUENCE LISTING (67 sequences)

SEQ ID NO: 1 - RNA, 19 nt, synthetic construct
  source 1..19: mol_type = other RNA
aggccacattcacgtatat

SEQ ID NO: 2 - RNA, 19 nt, synthetic construct
  source 1..19: mol_type = other RNA
tgaggaagtaccattatat

SEQ ID NO: 3 - RNA, 19 nt, synthetic construct
  source 1..19: mol_type = other RNA
ccggttgaatcttcagata

SEQ ID NO: 4 - RNA, 19 nt, synthetic construct
  source 1..19: mol_type = other RNA
gcgcaagtcctcaaagcac

SEQ ID NO: 5 - RNA, 18 nt, synthetic construct
  source 1..18: mol_type = other RNA
aggccacattcacgtata

SEQ ID NO: 6 - RNA, 18 nt, synthetic construct
  source 1..18: mol_type = other RNA
tgaggaagtaccattata

SEQ ID NO: 7 - RNA, 19 nt, synthetic construct
  source 1..19: mol_type = other RNA
aggccacattcacgtataa

SEQ ID NO: 8 - RNA, 21 nt, synthetic construct
  source 1..21: mol_type = other RNA
ttatacgtgaatgtggcctgt

SEQ ID NO: 9 - RNA, 19 nt, synthetic construct
  source 1..19: mol_type = other RNA
tgaggaagtaccattataa

SEQ ID NO: 10 - RNA, 21 nt, synthetic construct
  source 1..21: mol_type = other RNA
ttataatggtacttcctcaat

SEQ ID NO: 11 - RNA, 19 nt, synthetic construct
  source 1..19: mol_type = other RNA
atgaagtgtaccctaacta

SEQ ID NO: 12 - RNA, 21 nt, synthetic construct
  source 1..21: mol_type = other RNA
aagtctgcaacatggaaggta

SEQ ID NO: 13 - RNA, 19 nt, synthetic construct
  source 1..19: mol_type = other RNA
ggccacattcacgtatatg

SEQ ID NO: 14 - RNA, 21 nt, synthetic construct
  source 1..21: mol_type = other RNA
aagttcacctgagcctaatag

SEQ ID NO: 15 - RNA, 20 nt, synthetic construct
  source 1..20: mol_type = other RNA
actttcttggaaacgtgtaa

SEQ ID NO: 16 - RNA, 19 nt, synthetic construct
  source 1..19: mol_type = other RNA
gtcggacagcctcaccaaa

SEQ ID NO: 17 - RNA, 20 nt, synthetic construct
  source 1..20: mol_type = other RNA
agcgcaagtcctcaaagcac

SEQ ID NO: 18 - RNA, 16 nt, synthetic construct
  source 1..16: mol_type = other RNA
ccacattcacgtataa

SEQ ID NO: 19 - RNA, 21 nt, synthetic construct
  source 1..21: mol_type = other RNA
  misc_feature 1..3: note = modified with a phosphorothioate bond
  misc_feature 19..21: note = modified with a phosphorothioate bond
ttatacgtgaatgtggcctgt

SEQ ID NO: 20 - RNA, 21 nt, synthetic construct
  source 1..21: mol_type = other RNA
  misc_feature 1..3: note = modified with a phosphorothioate bond
  misc_feature 19..21: note = modified with a phosphorothioate bond
  misc_feature 7..8: note = modified with a phosphorothioate bond
ttatacgtgaatgtggcctgt

SEQ ID NO: 21 - RNA, 21 nt, synthetic construct
  source 1..21: mol_type = other RNA
  misc_feature 1..3: note = modified with a phosphorothioate bond
  misc_feature 19..21: note = modified with a phosphorothioate bond
  misc_feature 6..7: note = modified with a phosphorothioate bond
ttatacgtgaatgtggcctgt

SEQ ID NO: 22 - RNA, 21 nt, synthetic construct
  source 1..21: mol_type = other RNA
  misc_feature 1..3: note = modified with a phosphorothioate bond
  misc_feature 19..21: note = modified with a phosphorothioate bond
  misc_feature 5..6: note = modified with a phosphorothioate bond
ttatacgtgaatgtggcctgt

SEQ ID NO: 23 - RNA, 21 nt, synthetic construct
  source 1..21: mol_type = other RNA
  misc_feature 1..3: note = modified with a phosphorothioate bond
  misc_feature 19..21: note = modified with a phosphorothioate bond
  misc_feature 6..8: note = modified with a phosphorothioate bond
ttatacgtgaatgtggcctgt

SEQ ID NO: 24 - RNA, 21 nt, synthetic construct
  source 1..21: mol_type = other RNA
  misc_feature 1..3: note = modified with a phosphorothioate bond
  misc_feature 19..21: note = modified with a phosphorothioate bond
  misc_feature 5..7: note = modified with a phosphorothioate bond
ttatacgtgaatgtggcctgt

SEQ ID NO: 25 - RNA, 21 nt, synthetic construct
  source 1..21: mol_type = other RNA
  misc_feature 1..3: note = modified with a phosphorothioate bond
  misc_feature 19..21: note = modified with a phosphorothioate bond
  misc_feature 5..8: note = modified with a phosphorothioate bond
ttatacgtgaatgtggcctgt

SEQ ID NO: 26 - DNA, 24 bp, synthetic construct
  source 1..24: mol_type = other DNA
ctctgatcatctgaccaaaactca

SEQ ID NO: 27 - DNA, 21 bp, synthetic construct
  source 1..21: mol_type = other DNA
caacccagacatatccacctc

SEQ ID NO: 28 - DNA, 24 bp, synthetic construct
  source 1..24: mol_type = other DNA
  misc_feature 1: note = modified with a 5' 6-FAM
  misc_feature 9..10: note = modified with a ZEN quencher
  misc_feature 24: note = modified with a 3' Iowa Black FQ
tggcaagcatcctgtactgtcctg

SEQ ID NO: 29 - DNA, 19 bp, synthetic construct
  source 1..19: mol_type = other DNA
acatcgctcagacaccatg

SEQ ID NO: 30 - DNA, 22 bp, synthetic construct
  source 1..22: mol_type = other DNA
tgtagttgaggtcaatgaaggg

SEQ ID NO: 31 - DNA, 25 bp, synthetic construct
  source 1..25: mol_type = other DNA
  misc_feature 1: note = modified with a 5' 6-FAM
  misc_feature 9..10: note = modified with a ZEN quencher
  misc_feature 25: note = modified with a 3' Iowa Black FQ
aaggtcggagtcaacggatttggtc

SEQ ID NO: 32 - RNA, 16 nt, synthetic construct
  source 1..16: mol_type = other RNA
aagtgtaccctaacta

SEQ ID NO: 33 - RNA, 16 nt, synthetic construct
  source 1..16: mol_type = other RNA
tgcaacatggaaggta

SEQ ID NO: 34 - RNA, 16 nt, synthetic construct
  source 1..16: mol_type = other RNA
ccacattcacgtatat

SEQ ID NO: 35 - RNA, 16 nt, synthetic construct
  source 1..16: mol_type = other RNA
cacattcacgtatatg

SEQ ID NO: 36 - RNA, 16 nt, synthetic construct
  source 1..16: mol_type = other RNA
ggaagtaccattatat

SEQ ID NO: 37 - RNA, 16 nt, synthetic construct
  source 1..16: mol_type = other RNA
cacctgagcctaatag

SEQ ID NO: 38 - RNA, 16 nt, synthetic construct
  source 1..16: mol_type = other RNA
tcttggaaacgtgtaa

SEQ ID NO: 39 - RNA, 16 nt, synthetic construct
  source 1..16: mol_type = other RNA
gttgaatcttcagata

SEQ ID NO: 40 - RNA, 16 nt, synthetic construct
  source 1..16: mol_type = other RNA
caagtcctcaaagcac

SEQ ID NO: 41 - RNA, 16 nt, synthetic construct
  source 1..16: mol_type = other RNA
ggacagcctcaccaaa

SEQ ID NO: 42 - RNA, 17 nt, synthetic construct
  source 1..17: mol_type = other RNA
caagtcctcaaagcact

SEQ ID NO: 43 - RNA, 19 nt, synthetic construct
  source 1..19: mol_type = other RNA
tagttagggtacacttcat

SEQ ID NO: 44 - RNA, 21 nt, synthetic construct
  source 1..21: mol_type = other RNA
taccttccatgttgcagactt

SEQ ID NO: 45 - DNA, 21 bp, synthetic construct
  source 1..21: mol_type = other DNA
  misc_feature 1..19: note = RNA
  misc_feature 20..21: note = DNA
atatacgtgaatgtggccttt

SEQ ID NO: 46 - RNA, 19 nt, synthetic construct
  source 1..19: mol_type = other RNA
atatacgtgaatgtggcct

SEQ ID NO: 47 - RNA, 19 nt, synthetic construct
  source 1..19: mol_type = other RNA
catatacgtgaatgtggcc

SEQ ID NO: 48 - RNA, 19 nt, synthetic construct
  source 1..19: mol_type = other RNA
atataatggtacttcctca

SEQ ID NO: 49 - RNA, 21 nt, synthetic construct
  source 1..21: mol_type = other RNA
ctattaggctcaggtgaactt

SEQ ID NO: 50 - RNA, 20 nt, synthetic construct
  source 1..20: mol_type = other RNA
ttacacgtttccaagaaagt

SEQ ID NO: 51 - DNA, 21 bp, synthetic construct
  source 1..21: mol_type = other DNA
  misc_feature 1..19: note = RNA
  misc_feature 20..21: note = DNA
tatctgaagattcaaccggtt

SEQ ID NO: 52 - DNA, 21 bp, synthetic construct
  source 1..21: mol_type = other DNA
  misc_feature 1..19: note = RNA
  misc_feature 20..21: note = DNA
gtgctttgaggacttgcgctt

SEQ ID NO: 53 - DNA, 21 bp, synthetic construct
  source 1..21: mol_type = other DNA
  misc_feature 1..19: note = RNA
  misc_feature 20..21: note = DNA
tttggtgaggctgtccgactt

SEQ ID NO: 54 - DNA, 22 bp, synthetic construct
  source 1..22: mol_type = other DNA
  misc_feature 1..20: note = RNA
  misc_feature 21..22: note = DNA
gtgctttgaggacttgcgcttt

SEQ ID NO: 55 - RNA, 19 nt, synthetic construct
  source 1..19: mol_type = other RNA
aggccacattcacgtatat

SEQ ID NO: 56 - RNA, 16 nt, synthetic construct
  source 1..16: mol_type = other RNA
ggaagtaccattatat

SEQ ID NO: 57 - RNA, 16 nt, synthetic construct
  source 1..16: mol_type = other RNA
ggaagtaccattataa

SEQ ID NO: 58 - RNA, 16 nt, synthetic construct
  source 1..16: mol_type = other RNA
gttgaatcttcagatt

SEQ ID NO: 59 - RNA, 21 nt, synthetic construct
  source 1..21: mol_type = other RNA
atatacgtgaatgtggccttt

SEQ ID NO: 60 - RNA, 21 nt, synthetic construct
  source 1..21: mol_type = other RNA
ttatacgtgaatgtggccttt

SEQ ID NO: 61 - RNA, 21 nt, synthetic construct
  source 1..21: mol_type = other RNA
atataatggtacttcctcatt

SEQ ID NO: 62 - RNA, 21 nt, synthetic construct
  source 1..21: mol_type = other RNA
ttataatggtacttcctcatt

SEQ ID NO: 63 - RNA, 21 nt, synthetic construct
  source 1..21: mol_type = other RNA
ttataatggtacttcctcatc

SEQ ID NO: 64 - RNA, 19 nt, synthetic construct
  source 1..19: mol_type = other RNA
tatctgaagattcaaccgg

SEQ ID NO: 65 - RNA, 21 nt, synthetic construct
  source 1..21: mol_type = other RNA
tatctgaagattcaaccggtt

SEQ ID NO: 66 - RNA, 21 nt, synthetic construct
  source 1..21: mol_type = other RNA
aatctgaagattcaaccggtt

SEQ ID NO: 67 - RNA, 21 nt, synthetic construct
  source 1..21: mol_type = other RNA
aatctgaagattcaaccggcg